SEQUENCE LISTING (3 sequences)

SEQ ID NO: 1 - protein, 10 aa, Artificial Sequence
  peptide
DEHGTAVMLK

SEQ ID NO: 2 - protein, 4 aa, Artificial Sequence
  peptide
HMLK

SEQ ID NO: 3 - protein, 6 aa, Artificial Sequence
  peptide
HMHMHM

(19) United States
(12) Patent Application Publication
(10) Pub. No.: US 2024/0042001 A1
(43) Pub. Date: Feb. 8, 2024

(54) IRRADIATED WHOLE-CELL IMMUNOGENS OF ACINETOBACTER BAUMANNII

(71) Applicants: Biological Mimetics, Inc., Frederick, MD (US); The Henry M. Jackson Foundation for the Advancement of Military Medicine, Inc., Bethesda, MD (US)

(72) Inventors: Gregory J. Tobin, Frederick, MD (US); Taralyn J. Wiggins, Sharpsburg, MD (US); Michael J. Daly, Bethesda, MD (US); Elena Gaidamakova, Bethesda, MD (US); Vera Matrosova, Bethesda, MD (US)

(21) Appl. No.: 18/258,842

(22) PCT Filed: Dec. 21, 2021
(86) PCT No.: PCT/US2021/064605

§ 371 Date: Jun. 22, 2023

Related U.S. Application Data
(60) Provisional application No. 63/129,009, filed on Dec. 22, 2020.

(51) Int. Cl. A61K 39/02; C12N 1/36; C12N 1/20; A61P 31/04

(52) U.S. Cl. CPC A61K 39/0208; C12N 1/36; C12N 1/20; A61P 31/04; A61K 2039/522

(57) ABSTRACT

Presented herein are inactivated A. baumannii immunogens. Also described herein are compositions including A. baumannii immunogens. Methods and compositions for preparing the same are also described.

### STATEMENT OF PRIORITY

This application claims the benefit, under 35 U.S.C. § 119(e), of U.S. Provisional Application No. 63/129,009, filed on Dec. 22, 2020, the entire contents of which are incorporated by reference herein.

### STATEMENT REGARDING ELECTRONIC FILING OF A SEQUENCE LISTING

A Sequence Listing in ASCII text format, submitted under 37 C.F.R. § 1.821, entitled 1472-4WO_ST25.txt, 964 bytes in size, generated on Dec. 7, 2021, and filed via EFS-Web, is provided in lieu of a paper copy. This Sequence Listing is hereby incorporated herein by reference into the specification for its disclosures.

### BACKGROUND

The present invention relates to irradiation-inactivated A. baumannii bacterial immunogens and to compositions including the same and methods for preparing the same.

### BACKGROUND

The clinical treatment of infections from antibiotic-resistant bacteria is complex, expensive, and often ineffective. The continuous evolution of antibiotic resistance complicates the development of medical countermeasures. The availability of safe and effective vaccines against these types of pathogens would be of high value in preventing or mitigating infections.

Acinetobacter baumannii (A. baumannii) is a Gram-negative coccobacillus which is an opportunistic human pathogen that is listed by the World Health Organization (WHO) as a “top priority” pathogen for the development of novel and effective therapeutics and preventatives. A. baumannii carries robust antibiotic resistance mechanisms which include antibiotic-modifying enzymes, decreased permeability to antibiotic molecules, and active pumps that remove antibiotics into the periplasmic space. A. baumannii infections associated with hospital settings reflect the bacteria's ability to withstand the unfavorable conditions of a clinical environment. Due to the difficulties in treating and removing the threat of A. baumannii infections, a prophylactic vaccine is needed.

The design of vaccines against bacteria generally fall into two categories: (1) subunit vaccines and (2) whole-cell vaccines. Subunit vaccines, such as those for pertussis, pneumococcal, and meningococcal bacteria can be effective and their administration generally causes mild adverse reactions. However, the use of subunit vaccines generally requires many years or decades of research to identify the antigens of a bacterium that stimulate protective immunity. In addition, the manufacturing process by which recombinant proteins are expressed and purified requires considerable development to ensure that the proteins are produced in native form to stimulate protective immunity. For A. baumannii, no antigens have been identified and validated for a subunit based vaccine that stimulates protective immunity . Whole-cell vaccines, such as those for pertussis and anthrax often stimulate immunity with improved durability, but can cause more significant adverse reactions, especially at the site of immunization. Multiple strategies exist for the development of whole-cell bacterial vaccines including chemical inactivation, physical disruption, and irradiation. All three methods may produce a safe vaccine but also induce suboptimal immunity due to the disruption of antigenic epitopes during the inactivation process.

### SUMMARY OF THE INVENTION

The invention relates, in part, to novel whole-cell immunogens of A. baumannii which may have enhanced or novel immunogenicity. An A. baumannii immunogen composition of interest can serve as an immunogenic preparation and be used to produce antibodies, stimulate protective immunity from infection or disease, and/or to identify correlates of protective immunity.

Examples in this invention include compositions containing irradiation-inactivated (gamma ray and/or UVC inactivated) A. baumannii that stimulate immune responses for protection from disease and/or production of antibodies.

Embodiments of the present invention may produce compositions containing irradiation-inactivated (such as by gamma ray, x-ray, and/or UV (e.g., UVC)) A. baumannii which may improve the current practice of vaccine development by reducing damage to protective epitopes caused by chemical inactivation methods. In some embodiments, a protective antioxidant complex is used to reduce damage to protective epitopes during irradiation and the optimization of growth conditions that lead to the expression of protective antigens.

In some embodiments, inclusion of antioxidants such as manganese-peptide-orthophosphate (MDP) complexes may protect exterior macromolecules from damage during the radiation-inactivation process.

In some embodiments, the present invention provides a method by which novel immunogens of A. baumannii are designed and produced. The present invention may utilize a manganese-decapeptide-orthophosphate (MDP) complex to protect A. baumannii immunogens during supralethal irradiation thereby uncoupling cell death due to DNA damage from epitope destruction.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

The following drawings are provided to exemplify various aspects of the instant invention and are in no way to be interpreted as limiting the scope of the invention of interest.

FIG. 1 shows images of gels for Coomassie analysis of A. baumannii cells grown using varying conditions. Bacteria grown as either planktonic or biofilm forms were denatured and electrophoresed in an SDS-PAGE gel. P1: planktonic cells grown in a shaker or stirred culture in Tryptic Soy Broth (TSB) at 37° C. to stationary phase (approximately 16 to 24 hours), P2: planktonic cells grown in a shaker or stirred culture in TSB at 37° C. to logarithmic phase (approximately 2-6 hours), B1: biofilm grown submerged under M9 media in a plastic tissue culture flask at 37° C. for 3-7 days, B2: biofilm lawn grown on top of M9 agar at 37° C. for 1-3 days, B3: biofilm lawn grown on top of TSB media supplemented with sheep red blood cell lysate at 37° C. for 1-3 days. Lanes on the left side used 3 μl of loaded sample, lanes on the right used 12 μl of sample. Molecular weight (kDa) markers are shown on the left side of the figure. Arrows point to proteins of obvious increased or decreased expression based upon relative intensity.

FIG. 2 shows images of gels for Coomassie 2 Dimension (2D) electrophoresis analysis. A. baumannii proteins from biofilm culture (Biofilm 1) grown submerged under M9 minimal media (FIG. 2, Panel A) or in liquid planktonic cultures (Planktonic 2) until stationary growth phase (FIG. 2, Panel B) as described in FIG. 1. Arrows point to unique proteins or proteins that are expressed higher in one culture than the other.

FIG. 3 shows images of gels for a 2D immunoblot analysis. A. baumannii proteins from biofilm culture (Biofilm 1) grown submerged under M9 minimal media (FIG. 3, Panel A) or in liquid planktonic cultures (Planktonic 2) until stationary growth phase (FIG. 3, Panel B). The blots were developed using sera from mice immunized with the specific bacterial preparations.

FIG. 4 shows a graph of a survival curve for A. baumannii. Overnight planktonic A. baumannii was concentrated 5-fold and compounded with MDP to final cell concentrations of 5.42×10⁹ cfu/ml. Cells were then exposed to different doses of gamma-radiation (e.g., 0-10 kGy) and evaluated for surviving bacteria counts.

FIG. 5 shows a graph of an ultraviolet light inactivation curve for A. baumannii. Overnight planktonic A. baumannii was concentrated 5 times in MDP with a final cell concentration of 5.5×10⁹ cfu/mL. Cells were then exposed to UVC irradiation for varying lengths of time (e.g., 0-5 min) and then evaluated for surviving bacteria counts.

FIG. 6 shows graphs of example stability assays of nonirradiated and irradiated A. baumannii strain 5075 (AB5075) stored at 4° C. in either PBS or MDP. The bacteria were grown using the conditions described in FIG. 1. Nonirradiated samples of AB5075 were stored at 4° C. for varying lengths of time are shown in FIG. 6, panel A. At weekly or biweekly intervals, a portion of the sample was removed for colony counting by plating serial dilutions on agar plates. FIG. 6, panel B shows A. baumannii cells grown using varying conditions, complexed with MDP and exposed to 10 kGy gamma irradiation. The cells were stored at 4° C. for varying lengths of time between 0 and 16 weeks. At intervals, portions of the samples were removed for cell counting under a microscope using a hemocytometer. Undamaged cells were enumerated.

FIG. 7 shows graphs of survival of mice after immunization with irradiated A. baumannii and intranasal challenge. A. baumannii was propagated using a variety of methods as shown in FIG. 1. The bacterial samples were complexed with 1 mM MnCl₂, 3 mM DP1 peptide, 25 mM phosphate buffer and subjected to 10 kGy gamma irradiation. Portions of the samples equivalent to 33 10¹⁰ cells were plated on agar to ensure that the samples contained no residual replicative activity. Mice were immunized and boosted twice with 1×10⁷ cells and then challenged intranasally. FIG. 7, Panel A, shows immune competent C57BL/6 mice challenged with 1×10⁸ CFU of A. baumannii. FIG. 7, Panel B, shows Balb/c mice treated with cyclophosphamide prior to challenge with 5×10⁶ of A. baumannii. The graphs show surviving mice on each of the 7 days of the experiment.

FIGS. 8A-8B show graphs of survival of mice after immunization with irradiated A. baumannii and intranasal challenge. Gamma-irradiated A. baumannii from 16-h planktonic and/or biofilm grown under M9 media was used to immunize and boost twice mice. In the C57BL/6 mouse model (FIG. 8A), one of ten unimmunized mice survived pulmonary challenge to 7 days. In contrast, 100% of the mice immunized with either the biofilm grown under M9 or the combination preparation survived to Day 7. Eight of ten mice immunized with the 16h planktonic bacteria survived to Day 7. In the cyclophosphamide-treated Balb/c mouse model, one unimmunized mouse survived to Day 7 (FIG. 8B). Nine out of ten mice immunized with the combination survived to Day 7. Eight mice immunized with the 16-h planktonic and five immunized with the biofilm grown under M9 media survived to Day 7.

### DETAILED DESCRIPTION OF THE INVENTION

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

“Pharmaceutically acceptable” as used herein means that the compound, anion, cation, or composition is suitable for administration to a subject to achieve a treatment, such as one described herein, without unduly deleterious side effects in light of the severity of the disease and necessity of the treatment.

As used herein, the terms “increase,” “increases,” “increased,” “increasing,” “improve,” “enhance,” and similar terms indicate an elevation in the specified parameter of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more.

As used herein, the terms “reduce,” “reduces,” “reduced,” “reduction,” “inhibit,” and similar terms refer to a decrease in the specified parameter of at least about 5%, 10%, 15%, 20%, 25%, 30%, 35%, 40%, 45%, 50%, 55%, 60%, 65%, 70%, 75%, 80%, 85%, 90%, 95%, 97%, or 100%.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

The present invention provides methods by which A. baumannii immunogens are designed, produced, and/or obtained. In some embodiments, a method of the present invention may utilize a manganese-peptide-orthophosphate complex. Such a complex may protect a protein (e.g., an enzymatic protein) within the bacterium from oxidative damage caused by reactive oxygen species (ROS) that are formed during irradiation. A manganese-decapeptide-orthophosphate (MDP) complex of the present invention may be adapted from the bacterium Deinococcus radiodurans (D. radiodurans) for the protection of immunogens from ROS. The present invention may utilize MDP to protect immunogens from A. baumannii during supralethal irradiation, thereby uncoupling cell death due to DNA damage from epitope destruction.

Provided according to embodiments of the present invention are irradiation-inactivated A. baumannii immunogens. In some embodiments, the irradiation-inactivated immunogens of the present invention can stimulate antibodies that provide immune protection from infection and/or disease. An irradiation-inactivated A. baumannii immunogen of the present invention may stimulate protective immunity in a human (e.g., a human immunized with the immunogen).

“Immunogen” and “antigen” are used interchangeably herein and refer to a molecule that elicits a specific humoral and/or cellular-mediated immune response, for example, an immune response in which an antibody is stimulated and binds to the molecule or bacterium. The binding site for an antibody within an antigen and/or immunogen may be referred to as an epitope (e.g., an antigenic epitope).

A vaccine is an immunogen that is used to generate an immunoprotective response, e.g., by priming the immune system such that upon further exposure to an antigen (e.g., an immunogen and/or antigen of an infectious entity such as, e.g., an infectious bacterium) the immune response is more protective to the host (e.g., vaccine recipient, e.g., the subject) as compared to the immune response against exposure to the antigen without prior vaccination. For example, an induced antibody can be provided by a vaccine that reduces the negative impact of the immunogen found on an infectious bacterium, or entity expressing same, in a host. The dosage for a vaccine may be derived, extrapolated, and/or determined from preclinical and clinical studies, as known to those of skill in the art. Multiple doses of a vaccine may be administered as known in the art and/or may be administered as needed to ensure a prolonged prophylactic and/or anamnestic (memory) state (e.g., a primed state). In some embodiments, the successful endpoint of the utility of a vaccine for the purpose of this invention is the resulting presence of an induced immune response (e.g., humoral and/or cell-mediated) resulting, for example, in the production of serum antibody or antibodies made by the host which recognizes the intended antigen. Such antibodies can be measured as is known in the art by a variety of assays such as, e.g., neutralization assays of serum sampled from animals or humans immunized with said vaccine and/or immunogen.

Bacteria of use in the present invention include bacteria such as Acinetobacter baumannii. Non-limiting examples of A. baumannii strains and/or isolates of interest include, but are not limited to, AB5075, ABNIH2, ABNIH3, Naval-18, and AB058.

A. baumannii is a Gram-negative gamma proteobacteria which infects humans and/or other animals, sometimes leading to hospitalization and/or death. Multiple strains of A. baumannii are associated with antibiotic resistance and are difficult to treat with antibiotic therapies. There is no licensed vaccine against A. baumannii and therapeutic countermeasures to treat human infections are limited in both effectiveness and variety.

According to embodiments of the present invention, A. baumannii bacterial cells can be propagated in various ways to produce progeny cells that express varying protein profiles (e.g., immunogens). Cells propagated in liquid (e.g., liquid growth media) and collected from liquid are normally termed “planktonic” bacterial forms, while those propagated on solid substrate are normally termed “biofilm” forms. In some embodiments, a variety of growth media can be used including minimal nutrient and rich nutrient broths and agars. Biofilm forms can be grown on (e.g., above and/or underneath) surfaces of media (e.g., solid and/or liquid growth media). For example, biofilm forms can be grown on the surface of agar nutrient plates, on the inside surfaces of plastic tubing, on the surface of plastic plates underneath growth media, or using other methods known to the art. Cells grown using a variety of methods can be characterized by growth morphology, protein profiles, and/or other methods known to the art. Embodiments of the invention produce and/or provide a whole-cell A. baumannii bacterial vaccine and/or include a method for propagating A. baumannii bacteria such that the bacteria expresses proteins and/or other antigens that stimulate immune protection from later infection. For example, planktonic forms can be grown for about 2 to about 6 hours (e.g., about 2 hours, about 3 hours, about 4 hours, about 5 hours, about 6 hours or any value or range therein) in TSB media to exhibit logarithmic growth characteristics and/or for about 16 to about 36 hours (e.g., about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, or any value or range therein) in TSB to exhibit stationary growth phase characteristics. In further examples, biofilm forms can be grown for about 2 days to about 10 days (e.g., about 2 days, 3 days, 4 days, 5 days, 6 days, 7 days, 8 days, 9 days, 10 days, or any value or range therein) on plastic surfaces underneath liquid media, such as but not limited to M9 minimal media; for about 1 day to about 10 days (e.g., about 1 days, about 2 days, about 3 days, about 4 days, about 5 days, about 6 days, about 7 days, about 8 days, about 9 days, about 10 days, or any value or range therein) on the surface of agar plates using M9 media; for about 2 days to about 10 days (e.g., about 2 days, about 3 days, about 4 days, about 5 days, about 6 days, about 7 days, about 8 days, about 9 days, about 10 days, or any value or range therein) on plastic surfaces under rich media, such as but not limited to TSB; for about 2 days to about 10 days (e.g., about 2 days, about 3 days, about 4 days, about 5 days, about 6 days, about 7 days, about 8 days, about 9 days, about 10 days, or any value or range therein) inside plastic tubing using slowly flowing minimal media such as but not limited to M9 or 0.1×TSB; or other methods known to the art.

Growing A. baumannii bacteria under a variety of conditions may induce differential expression of virulence factors and/or bacterial antigens that stimulate a protective immune response.

Media used in embodiments of the present invention can be supplemented with materials of animal and/or human origin, including but not limited to sera, blood, synovial fluid, plasma, and/or brain extract, which may yield unique protein expression profiles (e.g., immunogens). Media supplements may be particularly relevant when microbes prefer proteins as a nutritional source or form biofilms in response to nutrients or scaffolds present in biological materials.

A. baumannii bacteria can be grown at different temperatures, different atmospheric oxygen, and/or different CO₂ concentrations, which may induce virulence factors and/or other regulatory events that may alter protein expression profiles (e.g., immunogens) including but not limited to temperatures of 72, 43, 40, 37, 32, 30, 28, 25, 23, 20, 17, 15, and 12° C., or any value or range therein.

A. baumannii bacteria can be grown in the presence of varying concentrations of gasses including low oxygen and/or high carbon dioxide concentrations. Oxygen can be varied to a range of concentrations including but not limited to about 0% to about 20% (e.g., about 0%, about 0.5%, about 1%, about 5%, about 10%, about 15%, about 17.5%, about 20%, or any value or range therein). CO₂ can be varied to a range of concentrations including but not limited to about 0% to about 15% (e.g., about 0%, about 1%, about 2%, about 5%, about 10%, about 12.5%, about 15%, or any value or range therein). Non-atmospheric gas concentrations can be achieved in a variable atmospheric incubator or by total or near total displacement of atmospheric gasses with heavier inert gasses.

A. baumannii bacteria can be grown and harvested at several time points yielding unique protein expression profiles. Time points may be designed to harvest bacteria from different growth phases ranging from lag, exponential, stationery (stable) and death phases of culture. Time points can include but are not limited to 30 minutes, 1 hour, 2 hours, 4 hours, 6 hours, 12 hours, 18 hours, 24 hours, 48 hours, 96 hours, 192 hours, 240 hours, or any value or range therein.

A. baumannii bacteria can be cultured using a variety of platforms to generate planktonic and/or biofilm forms with unique protein profiles (e.g., immunogens). Platforms include but are not limited to, continuous flow cultures such as tubing reactors, drip reactors, CDC tube reactors, inline reactors, annular reactors, and solid media plates (e.g., agar), shaking aqueous culture and stationary aqueous cultures.

Composite materials of bioreactor growth surfaces can be substituted to generate cultures with unique protein profiles (e.g., immunogens) including but not limited to, silicone, silicone-rubber, stainless steel, carbon steel, glass, polycarbonate, polypropylene, PVC, HDPE, polyurethane, nylon, rubber, titanium, iron, brass, bronze, nickel, concrete, hydroxyapatite and glass.

Cultures may be harvested and chilled to 4° C. to limit further growth and alteration of protein expression.

Cultures may be pelleted via centrifugation, and washed and resuspended in phosphate buffered saline (PBS) an optimal number of times (e.g., 2 times). This may serve to stabilize the protective effects of MDP upon irradiation (e.g., gamma and/or UVC) needed to preserve the integrity of the sample epitopes while removing nutrients to inhibit additional growth.

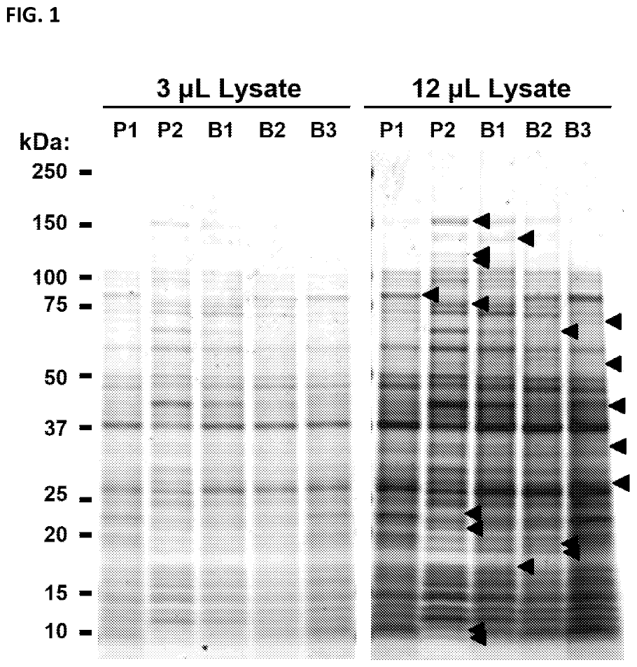

FIG. 1 shows a protein expression analysis of A. baumannii propagated in varying growth conditions herein defined as P1/planktonic 1: 16 h growth duration to stationary phase, 37° C. temperature, standard atmospheric gas environment, shaking aqueous culture, 1× Tryptic Soy Broth (TSB) nutrition; P2/planktonic 2: 2 h growth duration in logarithmic growth phase, 37° C. temperature, standard atmospheric gas environment, shaking aqueous culture, 1×TSB nutrition; B1/biofilm 1: 48 h growth duration, 37° C. temperature, standard atmospheric gas environment, hydrophobic polystyrene growth surface, growth surface submerged under stationary M9 media for nutrition (1× concentration); B2/biofilm 2: 24 h growth duration, 37° C. temperature, standard atmospheric gas environment, semi-solid 1% agar growth surface, agar surface formulated with M9 media for the nutritional component (1× concentration); and B3/biofilm 3: 24 h growth duration, 37° C. temperature, standard atmospheric gas environment, semi-solid 1% agar growth surface, agar surface formulated with TSB (1× concentration) supplemented with a 5% volume of sheep red blood cells for the nutritional component. The black arrows identify specific proteins that are expressed to a higher or lower level in one type of culture relative to another.

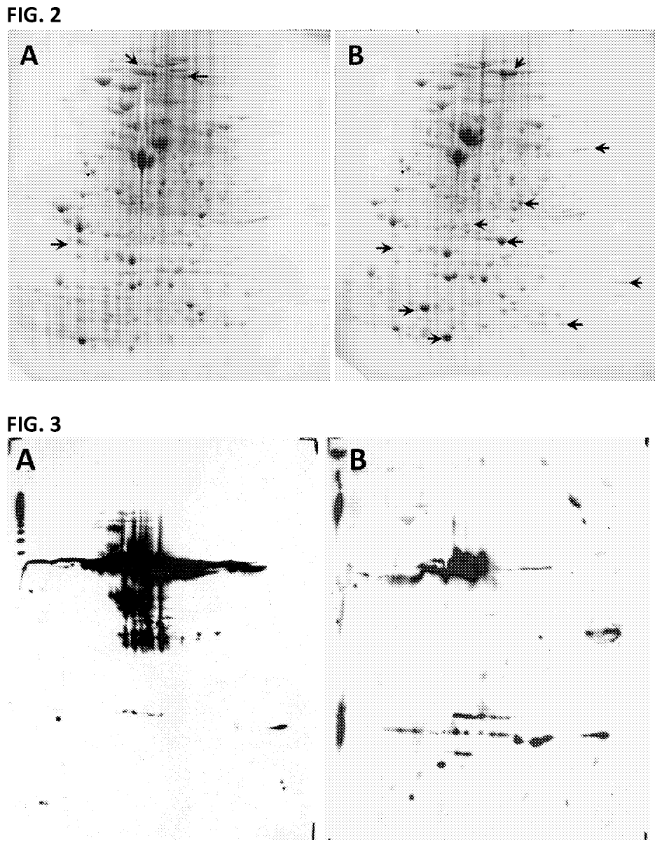

Methods known in the art, e.g., two-dimensional (2D) electrophoresis analysis, can be performed to identify proteins that are unique to a specific growth condition. For example, FIG. 2 shows a 2D gel for A. baumannii grown as a biofilm underneath minimal M9 media (e.g., FIG. 2 “biofilm 1”) and for A. baumannii grown for 16 hours in a shaking culture with Tryptic Soy Broth (TSB) (e.g., FIG. 2 “planktonic 1”) growth media. In this example analysis, proteins were denatured and separated by size in the first dimension and then by isoelectric point in the second dimension. The arrows point to proteins that are either unique to one culture or are over-expressed in one culture relative to the other.

Analysis of A. baumannii bacteria grown under differing conditions may also include 2D immunoblots. For example, FIG. 3 shows 2D immunoblots of gels similar in nature to those shown in FIG. 2. In both panels, bacterial culture samples were denatured, separated by size and then isoelectric point, and then transferred to membranes for immunoblotting. FIG. 3, panel A shows a 2D immunoblot of biofilm bacteria grown under M9 media. FIG. 3, panel B shows a 2D immunoblot of bacteria grown in a 16-hour shaking culture of TSB. Both immunoblots were probed with sera from rats immunized with B1 biofilm bacteria grown under M9 media. Many differences in the appearance and intensity of spots can be seen.

Additional analyses such as high-performance liquid chromatography (HPLC) and/or mass spectroscopy (MS), and/or other analyses known in the art may be performed to characterize the differences in protein profiles among samples of bacteria grown in varying conditions. Such characterizations may be useful for assessing the consistency of a whole-cell immunogen if it were manufactured as a vaccine. In addition, these methods are useful for identifying proteins that correlate with protective immunity.

A. baumannii bacteria (e.g., viable bacterial cells and/or radiation-inactivated bacterial cells) may be analyzed for stability, for example, by plating diluted samples for colony-forming units (CFU) counts on agar growth plates and/or by microscopic counting of cells, for example, in a hemocytometer.

Gamma (γ) rays, x-rays, and/or other types of radiations may be used in the sterilization of medical supplies and equipment. In cells, gamma- and x- radiation may cause direct damage by photons that indiscriminately introduce nicks into DNA, lesions into proteins, as well as lipid damage. However, the vast majority of radiation damage in aqueous conditions is indirect in nature and a result of reactive oxygen species (ROS) formed from the radiolysis of water. For example, the ROS superoxide (O₂^{⋅-})is particularly dangerous to proteins because of its selective reactivity with certain amino acids and with Fe²⁺ bound to proteins. Dismutation ofO₂^{⋅-} by Mn²⁺-peptide antioxidants may produce hydrogen peroxide (H₂O₂) that can escape cells through membranes, unlike O₂^{⋅-}.

A method of the present invention may use an antioxidant and/or antioxidant composition to protect antigenic epitope(s) on the surface of a bacterium, optionally while leaving the nucleic acid inside the bacterium subject to damage and/or destruction from ionizing radiation (e.g., gamma and/or x-ray radiation) and/or UV radiation (e.g., UVC radiation).

In some embodiments, a method of the present invention comprises providing an antioxidant composition (e.g., a composition comprising a peptide such as, e.g., a manganese-decapeptide-phosphate (MDP) composition) comprising a complex, which may protect A. baumannii bacterial epitopes during irradiation. An antioxidant composition of the present invention may comprise a divalent cation (e.g., Mn²⁺), a peptide, and a buffer system. In some embodiments, the antioxidant composition comprises manganese chloride (MnCl₂), a decapeptide, and a phosphate buffer. In some embodiments, the antioxidant composition comprises manganese chloride (MnCl₂), a decapeptide, and a Tris buffer. In some embodiments, the antioxidant composition comprises manganese chloride (MnCl₂), a decapeptide, and a 2-(N-morpholino)ethanesulfonic acid (MES) buffer. In some embodiments, an antioxidant composition of the present invention comprises a manganese-decapeptide-phosphate (MDP) complex.

The MDP complex may act as an antioxidant by preventing the accumulation of superoxide, which may limit the propagation of ROS. Manganese antioxidants like MDP are unique among redox active metal complexes accumulated in cells. Mn²⁺ ions are innocuous under conditions where other biologically active transition metals (e.g., Fe²⁺) tend to promote ROS; therefore, many cells can tolerate millimolar concentrations of Mn²⁺ within the cytoplasm. Moreover, Mn redox-cycling favors O₂^{⋅-}-scavenging without the release of extremely reactive hydroxy (HO^{⋅}) radicals. In contrast, the redox-cycling of Fe, Cr and Cu gives rise to HO^{⋅} radicals by Fenton-type reactions. Thus, without Mn antioxidants, O₂^{⋅-} radicals can become a significant source of HO^{⋅} radicals, and hence a significant factor in the biochemical mechanism of epitope damage during the preparation of irradiated immunogens and/or vaccines.

While not wishing to be bound by theory, in the presence of MDP, irradiation (e.g., gamma and/or x-ray irradiation) of A. baumannii may cause fewer oxidative lesions to proteins than when MDP is absent. MDP, however, may not protect nucleic acids (e.g., DNA and/or RNA). When A. baumannii is complexed with MDP, one or more immunogenic epitope(s) on the exterior of the bacterial cell (e.g., surface molecules) may be protected from ROS, optionally while the DNA inside the cell may be fragmented and/or oxidized. The end result may be an immunogenic cell that lacks replicative ability.

Compositions comprising MDP and A. baumannii bacteria of the present invention may be produced by combining and/or contacting (e.g., “complexing”) an amount of A. baumannii bacteria (e.g., 10⁵, 10⁶, 10⁷, 10⁸, 10⁹, 10¹⁰, 10¹¹, or other amounts of A. baumannii bacteria) with an amount of a divalent cation (e.g., manganese chloride), an amount of a peptide, and an amount of a buffer (e.g., a phosphate buffer).

In some embodiments, an antioxidant composition of the present invention may comprise a divalent cation, such as, e.g., manganous Mn²⁺. In some embodiments, the divalent cation may be provided as a salt, e.g., MnCl₂. In some embodiments, an antioxidant composition of the present invention may comprise a divalent cation (e.g., Mn²⁺) in a concentration of about 0.5 mM to about 10 mM, e.g., about 0.5, 1, 1.5, 2, 2.5, 3, 3.5, 4, 4.5, 5.5, 6, 6.5, 7, 7.5, 8, 8.5, 9, 9.5, or 10 mM, or any value or range therein. In some embodiments, a composition of the present invention may comprise a divalent cation in a concentration in the range of about 2 mM to about 5 mM (e.g., about 2, 2.5, 3, 3.5, 4, 4.5, or 5 mM or any value or range therein) . For example, in some embodiments, an antioxidant composition of the present invention may comprise Mn 2+ in an amount of about 1.4 mM to about 5.3 mM, about 2 mM to about 7 mM, or about 1 mM to about 9.8 mM. In some embodiments, an antioxidant composition of the present invention may comprise about 3 mM Mn²⁺. In some embodiments, an antioxidant composition of the present invention may comprise about 3 mM MnCl₂.

In some embodiments, an antioxidant composition of the present invention may comprise a peptide in a concentration of about 0.5 mM to about 10 mM (e.g., about 1, 1.5, 2, 2.5, 3, 3.5, 4, 4.5, 5, 5.5, 6, 6.5, 7, 7.5, 8, 8.5, 9, 9.5, 10 mM or any value or range therein). In some embodiments, an antioxidant composition of the present invention may comprise a peptide in a concentration in the range of about 2 mM to about 5 mM (e.g., about 2, 2.5, 3, 3.5, 4, 4.5, or 5 mM or any value or range therein). Thus, in some embodiments, the concentration of peptide (e.g., decapeptide) may be, for example, about 0.5 mM to about 5 mM, about 2 mM to about 7.5 mM, about 1.5 mM to about 8.5 mM, or about 2 mM, about 3.5 mM, about 3 mM, or about 5 mM in the composition.

In some embodiments, an antioxidant composition of the present invention may comprise a buffer, e.g., a phosphate buffer, a Tris buffer, an MES buffer, a HEPES buffer, and/or the like. The buffer may have a concentration of about 5 mM to about 500 mM (e.g., about 5, 10, 20, 30, 40, 50, 60, 70, 80, 90, 100, 150, 200, 250, 300, 350, 400, 450, 460, 470, 480, 490, 495, or 500 mM or any value or range therein). In some embodiments, the buffer may have a concentration in the range of about 25 mM to about 200 mM (e.g., about 25, 50, 75, 100, 125, 150, 175, or 200 mM or any value or range therein). Thus, in some embodiments, the concentration of buffer may be, for example, about 5 mM to about 450 mM, about 20 mM to about 500 mM, about 15 mM to about 350 mM, or about 25 mM, about 75 mM, about 200 mM or about 150 mM. In some embodiments, the buffer and/or antioxidant composition may have a pH of about 5 to about 9, or any value or range therein, e.g., about 6 to about 8.5, about 5 to about 7.8, about 6.5 to about 8, or about 6, about 6.8, about 7.4, or about 8.5. In some embodiments, an antioxidant composition of the present invention and/or method of their use may comprise a composition and/or method as described in PCT/US2008/073479; PCMS2011/034484; and/or PCMS2012/062998, the disclosures of which are incorporated herein by reference.

A peptide of the present invention may comprise 2 or more amino acids (e.g., 2, 3, 4, 5, 6, 7, 8, 9, 10, 11, 12, 13, 14, 15, 16, 17, 18, 19, 20, or more), optionally wherein the peptide comprises two or more amino acids residues from the sequence DEHGTAVMLK (SEQ ID NO:1) in any order and/or length. The exact sequence and/or length of the peptide may vary and the peptide may contribute to antioxidant activities and/or function as an antioxidant in a composition of the present invention. For example, in some embodiments the peptide may be a tetrapeptide (4mer), a pentapeptide (5mer), a hexapeptide (6mer), a heptapeptide (7mer), an octapeptide (8mer), a nonapeptide (9mer), and/or a decapeptide (10mer). The peptide may be manufactured synthetically in either the L- or D-configuration. In some embodiments, a peptide (e.g., a decapeptide) of the present invention may comprise the amino acids DEHGTAVMLK (SEQ ID NO:1) in any order and/or length, e.g., the peptide may comprise the sequence of amino acids HMLK (SEQ ID NO:2), a scrambled sequence of the amino acids HMLK (SEQ ID NO:2), the sequence of amino acids HMHMHM (SEQ ID NO:3), a scrambled sequence of the amino acids HMHMHM (SEQ ID NO:3), the sequence of amino acid DEHGTAVMLK (SEQ ID NO:1), and/or a scrambled sequence of the amino acids DEHGTAVMLK (SEQ ID NO:1). In some embodiments, a peptide may comprise an amino acid sequence having at least about 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence DEHGTAVMLK (SEQ ID NO:1). In some embodiments, a peptide may comprise an amino acid sequence having at least about 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence HMLK (SEQ ID NO:2). In some embodiments, a peptide may comprise an amino acid sequence having at least about 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence HMHMHM (SEQ ID NO:3).

The peptide may be a decapeptide with the amino acid composition of Asp-Glu-His-Gly-Thr-Ala-Val-Met-Leu-Lys (DEHGTAVMLK; SEQ ID NO:1). The peptide can be truncated to an amino acid composition of Asp-Glu-His-Gly-Thr-Ala-Val-Met or Asp-Glu-His-Met or similar, or rearrangements thereof. In some embodiments, the peptide may comprise an aspartic acid residue, a glutamic acid residue, a histidine residue, a glycine residue, a threonine residue, an alanine residue, a valine residue, a methionine residue, a leucine residue, and/or a lysine residue. In some embodiments, the peptide may comprise 1, 2, or more amino acid residues having a negatively charged side chain (e.g., aspartic acid and/or glutamic acid residues); 1, 2, or more amino acid residues having a positively charged side chain (e.g., histidine, lysine, and/or arginine residues); 1, 2, or more amino acid residues having a polar, uncharged side chain (e.g., threonine and/or serine residues); 1, 2, or more glycine residues; and/or 1, 2, 3, 4, or more amino acid residues having a hydrophobic side chain group (e.g., alanine, valine, methionine, leucine, and/or isoleucine residues). In some embodiments, the composition of amino acid residues in the peptide may be more important than the sequence of the amino acids.

A peptide of the present invention can be produced by enzymatic and/or chemical degradation of polypeptides such as casein, ovalbumin, whey, or other abundant and relatively inexpensive proteins. In some embodiments, the peptide contained within the MDP complex is not immunogenic and its inclusion with irradiated A. baumannii bacterial cells during injection into animals does not result in detected anti-peptide antibody production.

In some embodiments, an antioxidant composition of the present invention comprises MnCl₂ in a concentration of about 0.5 mM to about 10 mM, a decapeptide (e.g., DEHGTAVMLK [SEQ ID NO:1]) in a concentration of about 0.5 mM to about 10 mM, and a phosphate buffer in a concentration of about 5 mM to about 500 mM. In some embodiments, an antioxidant composition of the present invention comprises about 3 mM MnCl₂, about 3 mM decapeptide (e.g., DEHGTAVMLK [SEQ ID NO:1]), and about 200 mM phosphate buffer. However, concentrations of the components in the antioxidant composition may be varied as long as there is little degradation of effectiveness. An antioxidant composition may further comprise one or more excipient(s) such as, e.g., sorbitol, trehalose, etc., and/or one or more peptide(s) such as, e.g., HMHMHM (SEQ ID NO:3), HMLK (SEQ ID NO:2), and/or the like.

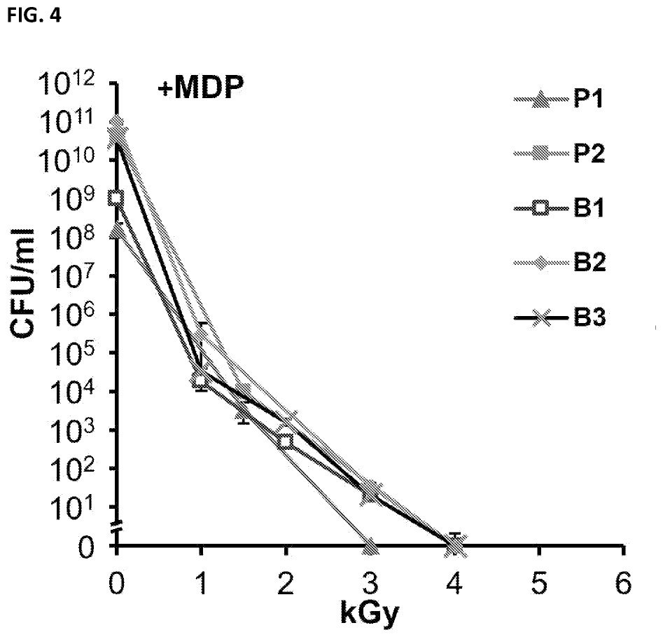

A peptide of the present invention can be assessed for suitability by performing a functional assay. FIG. 4 shows one example of a functional assay. Exposure to radiation (e.g., ionizing radiation such as gamma radiation, x-rays and/or ultraviolet light in the range of about 100 to about 400 nm and especially UVC in the range of about 200 to about 280 nm) can also be used to inactivate the replicative capability of A. baumannii bacterial cells.

A method of the present invention may comprise growing and/or culturing A. baumannii (e.g., planktonic and/or biofilm culture) to provide a cultured A. baumannii. The method may further comprise exposing A. baumannii (optionally in the presence or absence of an antioxidant composition, to radiation (e.g., ionizing (e.g., gamma and/or x-ray) radiation and/or ultraviolet (e.g., UVC) radiation), which may result in protection of one or more epitopes while leaving the A. baumannii bacterial genome exposed to damage and/or destruction from the radiation. In some embodiments, A. baumannii is exposed to ionizing radiation (e.g., gamma rays and/or x-rays) in an amount of at least about 0.5, 1, 0.5, 2, 2.5, 3, 3.5, 4, 4.5, 5, 6, 7, 8, 9, 10, 15, 20, 25, 30, 35, 40, 45, 50, 55, or 60 kGy, or any value or range therein. For example, in some embodiments, A. baumannii is exposed to ionizing radiation (e.g., gamma radiation and/or x-rays) in an amount of at least about 0.5 to about kGy, about 4 to about 9 kGy, about 1.5 to about 15 kGy, about 2 kGy to about 15 kGy, about 5 kGy to about 10 kGy, or about 1.5 kGy, about 4 kGy, about 7 kGy, about 8 kGy, or about 10 kGy or more. In some embodiments, A. baumannii is exposed to UV (e.g., UVC) radiation in an amount of about 0.01, 0.5, or 0.1 kJ/m² to about 5, 10, or 15 kJ/m² (e.g., about 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, or 15 kJ/m²), or an equivalent derived exposure time, surface area and/or light source wavelength and/or wattage. In some embodiments, A. baumannii is exposed to UV (e.g., UVC) radiation in an amount of about 0.01, 0.5, or 0.1 kJ/m² to about 0.5, 1, 1.5, 2, 2.5, 3, 3.5, 4, 4.5, 5, 5.5, or 6 kJ/m².

In some embodiments, A. baumannii may be exposed for about 60 minutes to a UVC light source emitting about 0.7 mW/cm², e.g., in an opaque plastic tube to destroy replicative activity. In some embodiments, A. baumannii is exposed for about 1, 5, 10, or 30 seconds to about 1, 1.5, or 2 minutes to a UVC light source emitting about 5 mW/c², e.g., when A. baumannii is contained in a UV-transparent vessel or tube also to destroy replicative activity. In some embodiments, A. baumannii may be exposed to a UV source (e.g., a UVC light source) having an intensity and/or for a period of time sufficient to inactivate the infectivity.

In some embodiments, a method of the present invention replaces air and/or dioxygen in contact with a composition of the present invention with argon. For example, the air in tubes comprising A. baumannii and optionally antioxidant composition may be at least partially replaced with argon. In some embodiments, a method of the present invention reduces the concentration and/or removes metals such as, e.g., iron, from compositions comprising A. baumannii and/or antioxidant composition. For example, the amount of trace iron contamination in phosphate buffers and other reagents may lead to increased oxidative damage of protein epitopes. Thus, in some embodiments, iron and/or other metals may be removed from buffers and water using methods known to those of skill in the art such as, e.g., by passage through a chelating chromatographic column (Chelex column, BioRad). In some embodiments, iron and/or other metals may be present in a concentration less than about 100 mM.

Provided according to some embodiments of the present invention is increased protection of A. baumannii bacterial epitopes (e.g., surface protein epitopes) from damage during the irradiation process (e.g., ionizing and/or ultraviolet irradiation). Increased protection of A. baumannii bacterial epitopes may be compared to a control, e.g., increased protection of epitopes during gamma and/or UVC irradiation inactivation as compared to formalin/formaldehyde inactivation. Increased protection may be accomplished by at least partially replacing ambient air with a non-reactive gas (e.g., argon) in containers (e.g., tubes) containing A. baumannii and/or removing and/or decreasing the amount of iron in compositions comprising the pre-inactivated A. baumannii. In some embodiments, air may be at least partially replaced with a non-reactive gas (e.g., argon). In some embodiments, air may be at least partially replaced with a non-reactive gas such that the content of oxygen is reduced by about 50% or more such as, e.g., by about 50%, 55%, 60%, 65%, 70%, 75%, 80%, 85%, 90%, 95%, 96%, 97%, 98%, 99% or more compared to the content of oxygen in the atmosphere and/or prior to the at least partial replacement.

Ultraviolet light may be used to inactivate A. baumannii with minimal to no damage to epitopes that stimulate neutralizing antibodies (e.g., antibodies against bacterial surface protein epitopes). Ultraviolet light can be divided into categories based on wavelength. UVA is 315-400 nm, UVB is 280-315 nm, and UVC is 100-280 nm. The infectivity of A. baumannii may be completely inactivated when exposed to a UVC (e.g., comprising a wavelength of about 220 to about 280 nm) light source emitting about 1 mW/cm² for about 60 minutes, e.g., in a partially opaque plastic tube, or a UVC light source emitting about 5 mW/cm² for about 1, 5, 10, or 30 seconds to about 1, 1.5, or 2 minutes if A. baumannii is contained in a UV-transparent vessel or tube.

As described herein, a method of the present invention may comprise exposing A. baumannii to radiation (e.g., ionizing and/or UV radiation) and the bacterium may be present in a vessel (e.g., a tube or container). As one of skill in the art would understand, the exposure conditions (e.g., intensity of radiation and/or time of exposure) may vary depending on the type of irradiation and the type and/or properties of the vessel. Ionizing radiation, such as gamma rays, are not easily blocked and the type of plastic or glass used in the vessel may not be critical. UVC radiation is more readily blocked by various types of plastic or glass. For example, in some embodiments, a vessel and/or tube may be clear and/or transparent, or may be opaque and/or frosted. In some embodiments, a vessel or tube may have a thickness of about 1 mm or more (e.g., about 1, 1.25, 1.5, 1.75, 2, 2.25, 2.5, 2.75, or 3 mm or more) (e.g., a “thick-walled” tube or vessel). In some embodiments, a vessel or a tube may have a thickness of about less than 1 mm (e.g., about 0.05, 0.1, 0.15, 0.25, 0.30, 0.35, 0.40, 0.45, 0.50, 0.55, 0.60, 0.65, 0.70, 0.75, 0.80, 0.85, 0.90, or 0.95 mm) (e.g., a “thin-walled” tube or vessel). In some embodiments, a method of the present invention may comprise exposing an immunogen of the present invention to radiation while the immunogen is flowing in and/or being transported through a vessel and/or tube (e.g., a flow cell).

Thus, in some embodiments, a method of the present invention may expose A. baumannii, optionally in a UV-transparent vessel or tube, to ultraviolet light (e.g., UVC) in an amount sufficient to at least partially inactivate the infectivity of the bacterium. In some embodiments, an amount sufficient to inactivate the A. baumannii may be a wavelength of about 220 to about 280, e.g., about 220, 225, 230, 235, 240, 245, 250, 255, 260, 265, 270, 275, or 280, or any range or value therein. In some embodiments, an amount sufficient to inactivate the A. baumannii may be a UVC light source emitting about 0.5 mW/cm² to about or 20 mW/cm² or more, e.g., about 0.5, 0.75, 1, 1.25, 1.5, 1.75, 2, 3, 4, 5, 6, 7, 8, 9, 10, 11, 12, 13, 14, 15, 16, 17, 18, 19, or 20 mW/cm² or any value or range therein. In some embodiments, an amount sufficient to inactivate the A. baumannii may be a UVC light source emitting about 10 mW/cm² or higher, e.g., wherein A. baumannii is highly concentrated, e.g., highly concentrated bacterial samples. While not wishing to be bound to theory, high-concentrated bacterial samples may require more UVC to inactivate potentially due to partial shielding of the light by the cells. In some embodiments, an amount sufficient to inactivate A. baumannii may be a UVC light source exposure for about 10 seconds to about 75 minutes, e.g., about 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, or 75 minutes, or any value or range therein. For example, in some embodiments, a method of the present invention may comprise exposing A. baumannii to UVC in an amount sufficient to completely inactivate the bacterium, e.g., about 30 minutes of exposure to a UVC light source at wavelength of about 254 emitting about 0.7 mW/c², or about 10 seconds to 5 minutes of exposure to a UVC light source at wavelength of about 254 emitting about 5 mW/cm² e.g., in a UVC-transparent tube or vessel. In some embodiments, when complexed with a MDP complex during UVC-inactivation A. baumannii epitopes are protected from damage as evidenced by stimulation of antibacterial antibodies and/or protective immune responses in animals or humans.

In some embodiments, a method of the present invention comprises exposing A. baumannii and optionally a MDP composition to ultraviolet (UV) light (e.g., UVC) and then to ionizing radiation.

The sterilizing effects of x-rays and/or gamma-rays in vaccine production are a result of direct damage to proteins and nucleic acids by photons and, more significantly (by far), indirect damage caused by reactive oxygen species (ROS) generated from the radiolysis of water molecules.

Some embodiments of the present invention result in protection of all or at least a portion (e.g., 10% or more, e.g., about 10%, about 20%, about 30%, about 40%, about 50%, about 60%, about 70%, about 80%, about 90%, about 95%, about 96%, about 97%, about 98%, or about 99% or more) of the exterior proteins that form the epitopes of the bacterium while leaving the RNA genome susceptible to destruction.

The potency of an A. baumannii bacterial vaccine or immunogen can be measured by analysis of the antibacterial immune activity in immunized humans or selected test animals, wherein higher quantification of antibacterial immune activity (e.g., bactericidal activity, e.g., bactericidal IgA, IgM and/or IgG, e.g., IgG1, IgG2, IgG3, and/or IgG4) activity in immunized subjects (e.g., humans and/or test animals) correlates with improved immunoprotective responses in subsequent exposure to wildtype bacteria. The potency of an A. baumannii bacterial vaccine or immunogen can be measured by analysis of the protective immunity raised in an immunized human or selected test animal that is challenged by either natural or experimental exposure to the bacterial pathogen. The quantitation of potency is measured using analyses known to the art which may include but are not limited to reduced bacterial burden in tissues, reduced disease parameters (e.g., reduced weight loss or behavioral signs), or reduced morbidity and/or mortality.

The irradiation-inactivated A. baumannii immunogens may be formulated in a simple solution such as water, a standard buffer, a standard saline solution, and/or the like. In some embodiments, an adjuvant may be included in a composition of the present invention, which may augment the magnitude and/or extend the duration of the immune response.

An immunogen and/or composition of the present invention may be provided and/or packaged in any suitable package and/or container. In some embodiments, an immunogen and/or composition of the present invention may be provided in a package suitable for administering the immunogen and/or composition to a subject. In some embodiments, glass vials, ampules, or other containers known to those of skill in the art may comprise an immunogen and/or composition of the present invention, optionally in single or multiple doses.

The amount of an immunogen administered to a subject and/or present in composition of the present invention is typically an amount sufficient to induce the desired immune response in the target host. Generally, the dosage employed may be about 0.1 microgram to about 100 micrograms of protein per dose (e.g., about 0.1, 0.2, 0.5, 1, 2, 3, 4, 5, 6, 7, 8, 9, 10, 15, 20, 25, 30, 35, 40, 45, 50, 55, 60, 65, 70, 75, 80, 85, 90, 95, or 100 micrograms of protein per dose, or any value or range therein).

The irradiation-inactivated A. baumannii immunogens of the present invention may be used to stimulate protective immunity in a subject (e.g., a human). The immunogens may be injected intramuscularly, intradermally, subcutaneously, and/or the like, into animals and/or humans, optionally using a standard syringe. In some embodiments, an immunogen of the present invention may be introduced into animals or humans using microneedles, patches designed to allow immunogens to penetrate the skin surface, and/or other methods known to the art.

In some embodiments, a manufacturing process for an immunogen of the present invention may include a procedure in which the immunogen is dried (e.g., desiccated by lyophilization, spray-drying, and/or the like). In some embodiments, the drying may increase the thermostability (e.g., by at least 10%, 15%, 20%, 25%, 30%, 35%, 40%, 45%, 50%, 55%, 60%, or more) of the immunogen and/or a composition comprising the immunogen and/or the drying may extend the shelf-life of the immunogen and/or a composition comprising the immunogen as measured (e.g., by at least 10%, 15%, 20%, 25%, 30%, 35%, 40%, 45%, 50%, 55%, 60%, or more), optionally by maintaining the immunogenic nature of the composition. The drying process may include compounding an immunogen of the present invention with one or more stabilizing excipient(s) known to those of skill in the art such as, but not limited to, sorbitol, trehalose, sucrose, polyethylene glycol, amino acids, and/or other additives. The drying procedure may utilize freeze-drying such as, e.g., lyophilization, spray-drying, and/or other methods known in the art.

In some embodiments, an adjuvant may be present in a vaccine of the present invention and the adjuvant may optionally stimulate an improved immune response. Example adjuvants include, but are not limited to, alum, aluminum hydroxide, aluminum phosphate, monophosphoryl Lipid A, saponin derivatives (e.g., QS-21), nucleic acids including oligonucleotides such as CpG, lipopolysaccharides, oil-and-water emulsions, squalene, saponin, and/or other adjuvanting substance(s) (e.g., flagellin).

The present invention is explained in greater detail in the following non-limiting examples.

### EXAMPLES

### Example 1: Gamma-Irradiation Inactivation of A. baumannii Leads to Partially Protective Immunogens

A. baumannii (“AB”) was propagated in planktonic and biofilm forms. Preliminary studies examined bacteria propagated in multiple types of media under a variety of conditions. After down-selection based on antigen profiles, five culture conditions were selected for analysis. Planktonic #1 and #2 cultures (P1 and P2) were grown in TSB media as shaker cultures at 37° C. for either 2 hours or 16 hours to produce cultures that are in either logarithmic growth or stationary phase, respectively. The biofilm forms were grown in a variety of manners known to the art. Biofilm #1 cultures (B1) were grown underneath M9 minimal media. 65 mL of M9 media was combined with 1 mL of stationary phase culture (P2) in in 225 cm² tissue culture flasks and incubated for 3-7 days at 37° C. Non-adherent cells were removed by changing the media every two days. At time of harvest, the culture media was replaced with 5 mL of cold PBS. The cells were removed from the surface using a plastic scraper. Biofilm #2 cultures (B2) were grown on the surface of M9 agar plates. 50 ul of P2 bacteria was spread on M9 agar plates which were incubated for 24-48 h at 37° C. At the time of harvest, 5 mL of cold PBS was added to each plate and the cells carefully scraped from the surface of the agar using a plastic scraper. Biofilm #3 culture (B3) was grown similarly to B2 except that the agar was comprised of TSB supplemented with 5% anti-coagulated sheep's blood. After each culture was harvested, the cells were pelleted by spinning at 5500×g for 15 minutes in a centrifuge at 4° C., decanting the liquid, and washing three times with ice cold PBS. Cells were stored at 4° C. in PBS at concentrations of 5×10⁹ to 2×10¹¹ CFU/mL.

Coomassie-stained protein gel analysis of these five samples are shown in FIG. 1. 100 ul volumes containing 1×10⁹ bacteria from the five samples were mixed with equal volumes of 4×SDS-PAGE sample buffer which contains 8% SDS, 2M 2-mercaptoethanol, 50 mM EDTA, and 40% glycerol in 200 mM Tris-HCl, pH 6.8. The samples were vortexed well, heated for 20 minutes at 65° C., vortexed again, and centrifuged at 17,000×g for 10 minutes to clarify the crude lysates. 3 ul (left side) or 12 ul (right side) were electrophoresed in denaturing polyacrylamide gels (4-20% acrylamide). After electrophoresis, the gels were stained in Coomassie Brilliant Blue R-250 and photographed. As can be seen in FIG. 1, the five different propagation methods produce bacteria that have five unique banding patterns. In the figure, protein bands that are present in higher concentrations in specific propagation samples are identified by small arrows.

Two-dimensional (2D) protein gels and blots can also provide helpful information in distinguishing differences between samples grown under different conditions. The 2D gels can help in quality control to ensure that batch-to-batch consistency is maintained. The 2D gels can also be used to correlate specific protein spots to protective immunity. When specific spots are present in a preparation that stimulates protection and are not in a preparation that does not stimulate protective immunity, these spots can be identified as correlating with protection. These analyses can also be used to identify proteins that could be used in subunit vaccines.

In the 2D analyses shown in FIG. 2, 5×10⁹ bacterial cells from the P1 (Panel A) and B1 (Panel B) samples were electrophoresed first in a denaturing gel that separates proteins by size (vertical direction) and then electrophocused to separate proteins by charge. The gel was Coomassie stained to visualize proteins. The spots that appear different in intensity between the two gels are located with arrows. Similarly, the 2D gels can be transferred to membranes and probed with serum from animals immunized with irradiation-killed bacteria. FIG. 3 shows examples of the P1 and B1 lysates analyzed by 2D western blot using sera from rats immunized with the B1 material. Again, many differences are apparent between the two samples. These differences can be correlated to differences in the stimulation of protective immunity.

Samples containing approximately 5×10⁹ CFU of bacteria grown using the five conditions above were complexed with 1 mM MnCl₂, 3 mM DP-1, and 25 mM potassium phosphate buffer, pH 7.4.

Closed test tubes containing the A. baumannii-MDP complex were placed on ice and introduced into an irradiation chamber. The dose of gamma, x-ray, UVC or other irradiation required to inactivate the replicative ability of the bacteria was assessed by spreading the bacteria onto agar plates containing nutrient media, incubating the plates at 37° C. for 16-36 hours, and counting the colonies. The colony counts were expressed as colony forming units (CFU) (e.g., 10¹⁰ CFU). A kill curve was performed using doses ranging from 0 to 10 kGy. The minimum dose of irradiation required to kill 100% of the bacteria can be determined in a small number of experiments. In many cases, doses of 6, 7, 8, or 9 kGy kill (sterilize) 100% of the bacteria in samples of 10⁹, 10¹⁰, 10¹¹ or 10¹² cells.

The data in FIG. 4 demonstrate a typical gamma irradiation kill-curve. Bacterial samples exposed to a dose of 4 kGy or greater showed no colony growth on the test plates indicating that 100% of the cells were rendered non-replicative.

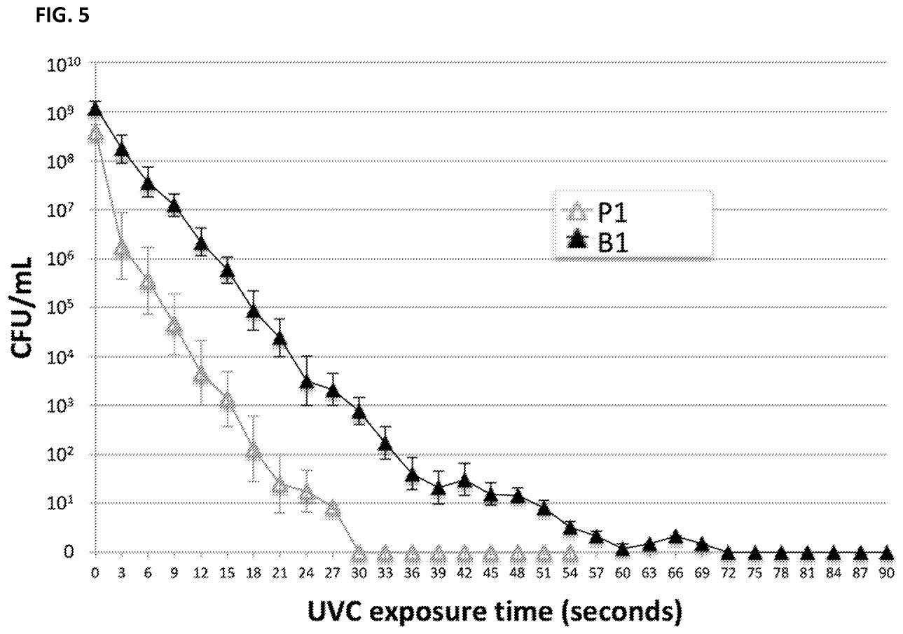

FIG. 5 shows a survival curve for A. baumannii cultures exposed to increasing doses of UVC light (254 nm) A. baumannii cultures B1 and P1 were propagated using the methods shown in the samples in FIG. 4 and complexed with MDP using 1 mM MnCl₂, 3 mM DP1 (DEHGTAVMLK; SEQ ID NO:1) decapeptide, and 25 mM potassium phosphate buffer (pH 7.4). 0.1 mL bacterial samples between 5×10⁸ and 2×10⁹ CFU/ml were exposed to 4.5 mW/cm2 UVC irradiation. After 30 seconds, the P1 culture contained no detectable live bacteria. After 72 seconds, the B1 culture contained no detectable live bacteria.

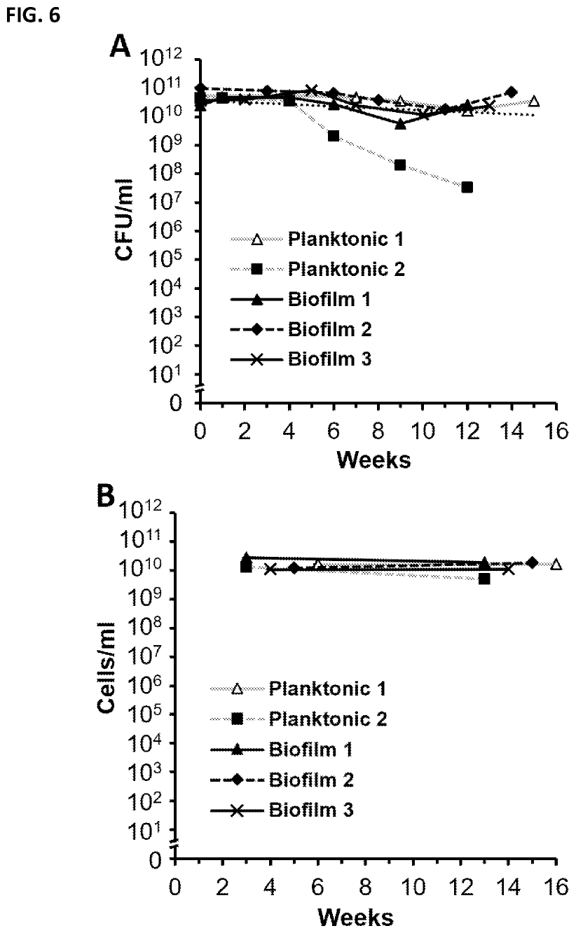

Stability assays were conducted to determine shelf-life of the bacterial preparations. Samples that were either not irradiated (FIG. 6, panel A) or irradiated with 10 kGy (FIG. 6, panel B) were stored at 4° C. At varying time points, the non-irradiated samples were analyzed for viability of the bacteria by plating serial dilutions of the sample onto agar plates and enumerating CFU the next day to determine the number of live cells per mL. With the exception of the samples from logarithmic planktonic growth conditions, the various preparations maintained the starting viability for at least 14 weeks (FIG. 6, panel A). The irradiation-killed samples were analyzed during the same time course by counting bacteria microscopically. A similar result was obtained (FIG. 6, panel B). FIG. 6 shows the stability of A. baumannii after storage at 4° C. for varying lengths of time.

Five samples shown in FIG. 4 were selected for immunization and challenge experiments as described below. In addition, a sixth sample (herein termed “cocktail”) was composed of an equal quantity of the five samples, B1, B2, B3, P1, and P2. The bacteria were complexed with MDP and then exposed to supralethal gamma irradiation (8-10 kGy).

Two mouse models were used for analysis of the immunogenicity. In both cases, mice were immunized and boosted twice with 1×10⁷ inactivated bacterial cells. C57/BL6 mice are susceptible to infection by A. baumannii using a variety of routes. Balb/c mice are less susceptible. However, Balb/c mice pre-treated with cyclophosphamide after immunization and before challenge are of heightened susceptibility and are among the most sensitive to infection and disease.

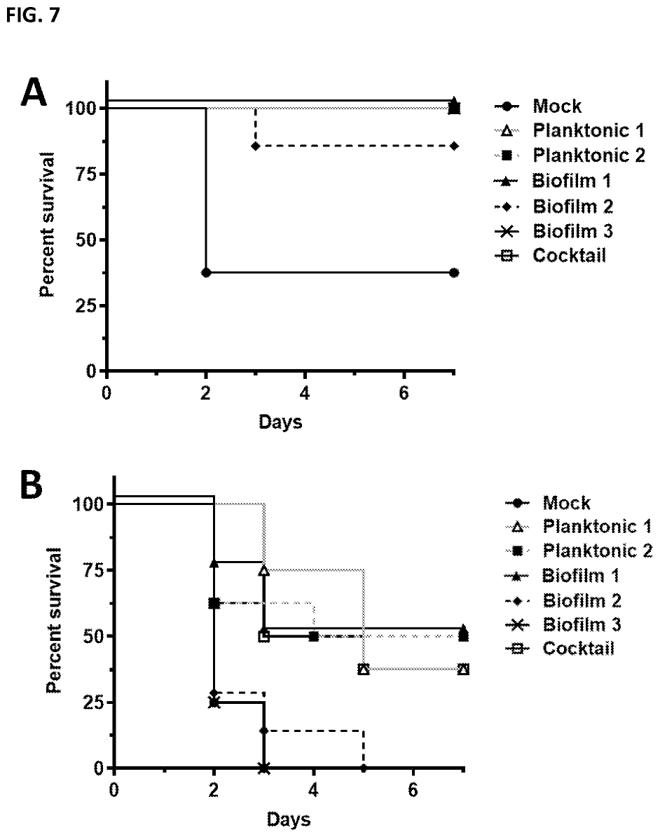

FIG. 7, panel A shows the survival plots of C57/BL6 mice using group sizes of eight. After challenge, three of the eight mice in the mock antigen (negative control) immunization group survived through Day 7. In contrast, eight of the mice in all of the immunization groups except B2 survived the pulmonary challenge.

FIG. 7, panel B shows the survival plots of BALB/c mice that were treated with cyclophosphamide prior to challenge in an effort to reduce neutrophil activity and, thereby, increase their sensitivity to infection and disease. In this experiment 100% of the mice that were not immunized died within three days. 100% of the mice immunized with either the biofilm grown on M9 (biofilm 2) or TSB (biofilm 3) plates died within five days. In contrast, groups of mice immunized with the two planktonic (planktonic 1 and 2), the biofilm grown under M9 media (biofilm 1), or the combination immunogen (cocktail) contained survivors out to seven days.

The results shown in FIG. 7, panels A and B were used to down-select immunogens for future studies. Follow-up studies compared the protective immunogenicity of (1) planktonic shaking culture grown for 16 hours (P1), (2) biofilm culture grown under M9 media (B1), and (3) a combination of 16-hour planktonic and M9 biofilm samples (P1+B1). The experiment again used the two mouse models: (a) immune competent C57BL/6 and (b) neutropenic Balb/c mice infected through the pulmonary route.

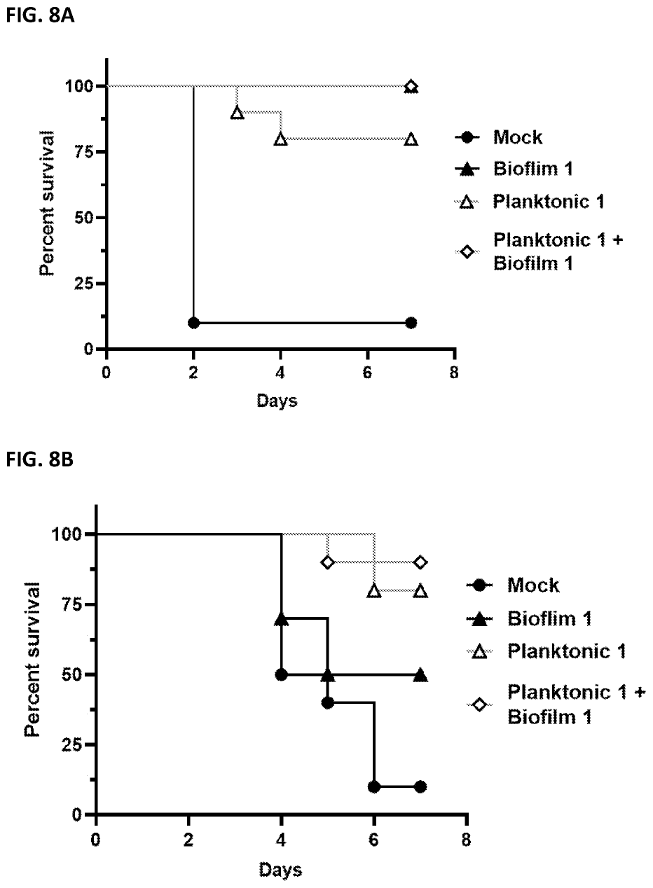

Groups of 10 mice were immunized and boosted twice with 1×10⁷ gamma-irradiated A. baumannii. Three weeks after the final boost, the mice were challenged with 1×10⁷ freshly-prepared, live A. baumannii. FIG. 8A shows the survival curves for the immune competent C57/BL6 mice and FIG. 8B the curves for the cyclophosphamide-treated Balb/c mice. In both cases, nine of ten unimmunized mice died by Day 7. Also in both cases the groups immunized with the combination of the 16-hour planktonic and the biofilm grown under M9 media showed the greatest rates of survival (100% and 90%, respectively). These data demonstrate the power of selecting immunogens based upon the protein profiles after growing under various conditions.

### Example 2: Protection in a Skin Wound Model

A. baumannii infects humans through a variety of routes including pulmonary, sepsis, and skin wounds. The studies shown above in FIGS. 6 and 7 modeled the pulmonary or inhalation route of infection. Mouse models of skin wounds can also assess the protective immunity of an irradiation-inactivated A. baumannii. In the skin wound model, mice are anesthetized, a patch of hair is gently removed from the back, and a disk of skin (approximately 5 mm in diameter) is removed using a skin punch device. Without infection, the wound heals in approximately 14-16 days. When A. baumannii is introduced into the wound, healing takes approximately 28 days. In addition to measuring the rate of wound closure and healing, a challenge strain that has been engineered to produce bioluminescence can be used. In the latter case, the progress of bacterial growth and clearance can be quantitated in the first week after challenge using a light measuring device.

To prepare bacteria for immunization, A. baumannii cultures were grown as described above to produce P1 (stationary planktonic grown in TSB) and B1 (biofilm grown under the surface of M9 media) cultures. The bacteria were washed with PBS and complexed with MDP as described above. Portions of each of the two preparations were inactivated using 10 kGy gamma irradiation, as described above. After testing portions of the gamma and UVC-inactivated bacteria to ensure that no residual replicative activity remained, the samples were prepared for immunization.

Groups of 10 Balb/c mice were immunized with (1) mock immunogen, (2) gamma-inactivated 16-hour planktonic bacteria, (3) gamma-inactivated biofilm grown under M9, or (4) a combination of the gamma-inactivated planktonic and biofilm.

### Example 3—Comparison of UVC and Gamma Irradiation in Both Models

The replicative capacity of A. baumannii can be inactivated by exposure to gamma irradiation or UVC light as shown above in FIGS. 4 and S.

The ability for UVC- and gamma-inactivated A. baumannii bacteria to stimulate protective immunity in both the pulmonary and skin wound models was compared. As in the previous example, two preparations of A. baumannii were propagated: P1 (planktonic grown in TSB) and B1 (biofilm grown under M9). Samples of P1 and B1 were complexed with MDP as described above. Samples were inactivated with 10 kGy gamma radiation on ice or 100 mJoules UVC light in thin-walled PCR tubes at concentrations of 5×10⁹ cfu/mL. After confirming that no residual CFU-forming activity remained in the irradiated samples, portions of the immunogens were combined to produce the P1+B1 immunogen.

Groups of 10 mice are immunized and boosted twice with 1×10⁷ irradiation-inactivated A. baumannii cells and then challenged intranasally as in the above examples. Survival studies are performed in the mouse pulmonary model. Bacterial load in the wound areas is measured in the skin infection model.

That which is claimed is:

1. An A. baumannii immunogen, wherein the immunogen is obtained and/or derived from irradiation-inactivated A. baumannii bacteria and/or wherein the immunogen is irradiation-inactivated.

2. The A. baumannii immunogen of claim 1, wherein the A. baumannii immunogen is obtained and/or derived from irradiation-inactivated A. baumannii grown under conditions such that one or more different bacterial immunogens that stimulate protective immunity are present, optionally under conditions such that two or more different bacterial immunogens that stimulate protective immunity are present.

3. The A. baumannii immunogen of claim 1 or 2, wherein the immunogen has been inactivated using ionizing radiation (e.g., gamma irradiation and/or x-ray irradiation).

4. The A. baumannii immunogen of any one of claims 1-3, wherein the A. baumannii immunogen has been inactivated using ultraviolet light irradiation (e.g., UVC radiation, optionally having a wavelength of about 180 nm to about 280 nm (e.g., about 180, 190, 200, 210, 220, 230, 240, 250, 260, 270, or 280 nm or any value or range therein, e.g., about 254 nm)).

5. The A. baumannii immunogen of any one of claims 1-4, wherein the bacteria are complexed (e.g., combined and/or contacted) with an antioxidant (e.g., manganese-decapeptide-orthophosphate (MDP)), optionally wherein the A. baumannii immunogen is complexed with an antioxidant prior to exposure to radiation (e.g., ionizing radiation and/or UVC radiation).

6. The A. baumannii immunogen of any one of claims 1-4, wherein the A. baumannii immunogen is not complexed (e.g., combined and/or contacted) with an antioxidant (e.g., manganese-decapeptide-orthophosphate (MDP)) and/or has been irradiation-inactivated in the absence of an antioxidant.

7. The A. baumannii immunogen of any one of claims 1-6, wherein the A. baumannii immunogen has been isolated (e.g., purified) from a culture of irradiation-inactivated A. baumannii.

8. The A. baumannii immunogen of claim 7, wherein the culture is a planktonic culture or a biofilm culture.

9. A composition comprising an A. baumannii immunogen of any of the preceding claims.

10. An immunogenic composition comprising an attenuated strain of A. baumannii expressing an antigenic epitope-containing bacterial protein, wherein bacterial infectivity of the A. baumannii has been decreased or abolished by ionizing radiation and/or UV radiation.

11. The immunogenic composition of claim 10, wherein the attenuated strain of A. baumannii expressing an antigenic epitope-containing bacterial protein is prepared by a method comprising:
exposing A. baumannii to ionizing radiation (e.g., x-ray and/or gamma-ray) and/or ultraviolet radiation (e.g., UVC) in an amount sufficient to inactivate the A. baumannii, thereby decreasing or abolishing the bacterial infectivity of the A. baumannii to provide irradiation-inactivated A. baumannii bacteria.

12. The immunogenic composition of claim 10 or 11, wherein the antigenic epitope-containing bacterial protein is protected from damage using a chemical complex comprising a manganous ion (Mn²⁺), a peptide, and a buffer (e.g., a phosphate and/or Tris and/or MES and/or HEPES buffer).

13. The immunogenic composition of claim 12, wherein the attenuated strain of A. baumannii expressing an antigenic epitope-containing bacterial protein is prepared by a method comprising:
contacting a complex comprising a manganous ion (Mn²⁺), a peptide, and a buffer (e.g., a phosphate and/or Tris and/or MES and/or HEPES buffer) with the A. baumannii prior to the exposing step;
thereby protecting the antigenic epitope-containing bacterial protein from damage.

14. The immunogenic composition of any one of claims 10-13, wherein the composition has been dried (e.g., lyophilized, spray-dried, or heat-dried).

15. The immunogenic composition of claim 14, wherein the composition is prepared by a method comprising:
drying (e.g., lyophilizing, spray-drying, heat-drying) the inactivated immunogenic composition.

16. The composition of claim 9 or the immunogenic composition of any one of claims 10-15, further comprising alum, aluminum hydroxide, aluminum phosphate, monophosphoryl lipid A, squalene, saponin and/or derivatives thereof (e.g., QS-21), flagellin, CpG, lipopolysaccharide, and/or oil-and-water emulsion.

17. A vaccine comprising an A. baumannii immunogen of any one of claims 1-8, a composition of claim 9 or 16, or an immunogenic composition of any one of claims 10-16.

18. Use of the A. baumannii immunogen of any one of claims 1-8, the composition of claim 9 or 16, or the immunogenic composition of any one of claims 10-16 to stimulate protective immunity from an infection.

19. Use of the A. baumannii immunogen of any one of claims 1-8, the composition of claim 9 or 16, or the immunogenic composition of any one of claims 10-16 to derive a reagent useful in the study of Acinetobacter.

20. Use of the A. baumannii immunogen of any one of claims 1-8, the composition of claim 9 or 16, or the immunogenic composition of any one of claims 10-16 to derive a reagent useful in the analysis of a mammalian (e.g., human, veterinarian) infection.

21. A method of producing an inactivated A. baumannii immunogen, the method comprising:
exposing A. baumannii bacteria to ionizing radiation and/or ultraviolet radiation in an amount sufficient to inactivate the A. baumannii bacteria, thereby providing the inactivated A. baumannii immunogen.

22. The method of claim 21, further comprising, prior to the exposing step, culturing A. baumannii to obtain cultured A. baumannii.

23. The method of claim 21 or 22, wherein the exposing step comprises exposing the cultured A. baumannii to ionizing radiation (e.g., x-ray and/or gamma-ray) and/or ultraviolet radiation (e.g., UVC)).

24. The method of any one of claims 21-23, wherein the culturing step comprises growing the A. baumannii using planktonic growth conditions, optionally wherein the culturing step comprises growing the A. baumannii for about 2 hours to about 6 hours to stationary phase and/or about 16 to about 24 hours to logarithmic phase, optionally in TSB media at 37° C.).

25. The method of any one of claims 21-23, wherein the culturing step comprises growing the A. baumannii using biofilm growth conditions, optionally wherein the culturing step comprises growing the A. baumannii for about 1 day to about 7 days, optionally in and/or on M9 media (e.g., wherein the A. baumannii are grown submerged in M9 media in liquid form and/or grown on top of M9 media in agar form), further optionally wherein the M9 media is supplemented (e.g., with sheep red blood cell lysate).

26. The method of any one of claims 21-25, wherein exposing the A. baumannii or cultured A. baumannii to ionizing radiation comprises exposing the A. baumannii to ionizing radiation in an amount of about 8 to about 10 kGy or more and/or wherein exposing the A. baumannii or cultured A. baumannii to ultraviolet radiation comprises exposing the A. baumannii for about 5 minutes to a UVC lamp emitting about 3 mW/cm², optionally in a thin-walled tube.

27. The method of any one of claims 21-26, further comprising the step of:
exposing the A. baumannii or cultured A. baumannii to a divalent cation (e.g., M²⁺), a peptide (e.g., a decapeptide), and a buffer or a complex thereof, prior to exposing the A. baumannii or cultured A. baumannii to ionizing radiation and/or ultraviolet radiation.

28. The method of claim 27, wherein exposing the A. baumannii or cultured A. baumannii to the divalent cation, peptide, and buffer comprises combining and/or contacting (e.g., complexing) the A. baumannii with a composition comprising the divalent cation, peptide, and buffer to provide a combined composition.

29. The method of claim 27 or 28, wherein the divalent cation is manganous, optionally wherein the divalent cation is provided by manganese chloride.

30. The method of any one of claims 27-29, wherein the peptide is HMLK (SEQ ID NO:2), HMHMHM (SEQ ID NO:3), and/or DEHGTAVMLK (SEQ ID NO:1).

31. The method of any one of claims 27-30, wherein the buffer comprises a phosphate buffer, optionally a potassium phosphate buffer.

32. The method of any one of claims 28-31, wherein the composition comprises MnCl₂ in a concentration of about 0.5 mM to about 10 mM, the peptide (e.g., HMLK [SEQ ID NO:2], HMHMHM [SEQ ID NO:3], and/or DEHGTAVMLK [SEQ ID NO:1]) in a concentration of about mM to about 10 mM, and a phosphate buffer (e.g., pH 7.2) in a concentration of about 5 mM to about 500 mM.

33. The method of any one of claims 21-32, wherein the exposing step comprises exposing the A. baumannii or cultured A. baumannii to ionizing radiation and then exposing the A. baumannii or cultured A. baumannii to ultraviolet radiation (e.g., UVC), optionally in an amount sufficient to at least partially inactivate the A. baumannii or cultured A. baumannii.

34. The method of any one of claims 21-33, further comprising, prior to exposing the A. baumannii or cultured A. baumannii to ionizing and/or ultraviolet radiation, at least partially replacing air in contact with the A. baumannii and/or in a container comprising the A. baumannii with a non-reactive gas (e.g., argon), optionally wherein at least partially replacing air comprises reducing the content of oxygen by about 50% or more.

35. The method of any one of claims 28-34, further comprising reducing an amount of iron present in the composition and/or in the combined composition.

36. The method of any one of claims 28-36, wherein the composition and/or combined composition further comprises one or more excipients and/or a peptide (e.g., HMHMHM [SEQ ID NO:3], HMLK [SEQ ID NO:2], and/or the like).

37. The method of any one of claims 21-36, further comprising drying the inactivated A. baumannii immunogen, optionally freeze-drying (e.g., lyophilizing) and/or spray-drying the inactivated A. baumannii immunogen.

38. The method of any one of claims 21-37, wherein the A. baumannii immunogen comprises one or more epitopes (e.g., neutralizing epitopes) and at least a portion of the one or more epitopes are protected, optionally wherein the at least a portion of the one or more epitopes are protected by a divalent cation, peptide, and buffer, during the step of exposing the A. baumannii or cultured A. baumannii to radiation.

39. The method of claim 38, wherein the at least a portion of one or more epitopes (e.g., neutralizing epitopes) are undamaged and/or active (e.g., stimulate neutralizing antibodies) in the inactivated A. baumannii immunogen.

40. An immunogen prepared or obtained from a method of any one of claims 21-39.

41. Use of the A. baumannii immunogen of any one of claims 1-8, the composition of any one of claims 9-16, the A. baumannii immunogen produced according to any one of claims 21-39, or the immunogen of claim 41 to derive reagents useful in the study of Acinetobacter and/or diagnostics useful in the analysis of potential human or animal infections.
